(19) United States
(12) Patent Application Publication
(10) Pub. No.: US 2004/0265375 A1
(43) Pub. Date: Dec. 30, 2004

(54) Orally disintegrating tablets

(76) Inventors: Johannes J. Platteeuw, 's-Hertogenbosch (NL); Dennie J. M. van den Heuvel, Boxmeer (NL)

Correspondence Address: MARK R. BUSCHER, P.O. BOX 161, CATHARPIN, VA, 20143, US

(21) Appl. No.: 10/824,619

(22) Filed: Apr. 15, 2004

Related U.S. Application Data
(60) Provisional application No. 60/463,027, filed on Apr. 16, 2003.

(51) Int. Cl. A61K 9/20

(52) U.S. Cl. 424/464; 514/57

(57) ABSTRACT

Silicified microcrystalline cellulose is used to provide a tablet with oral disintegration. The tablet contains at least 50% of the silicified microcrystalline cellulose and an effective amount of an active agent, especially a pharmaceutically active agent.

This application claims the benefit of U.S. provisional patent application Ser. No. 60/463,027, filed Apr. 16, 2003, the entire contents of which are incorporated herein by reference.

### BACKGROUND OF THE INVENTION

The present invention relates to orally disintegrating dosage forms that contain silicified microcrystalline cellulose.

Orally disintegrating dosage forms for delivery of pharmaceuticals are known in the art. The purpose of such systems is to allow administration of a solid dosage form, for instance a tablet, of a beneficial drug to a patient without the need to swallow the dosage form. The orally disintegrating tablet should disintegrate and, optionally dissolve, directly in oral cavity, with the aid of saliva or, in some cases a small amount of water. The resulting liquid or dispersion is then easily swallowed. This causes easy and immediate entry of the dissolved or dispersed beneficial drug into the gastrointestinal tract. In some cases the drug may even be absorbed by the oral mucosa or the esophageal lining as it passes down to the stomach. Orally disintegrating tablets, contrary to candies or sublingual tablets, should disintegrate in a time not exceeding one minute or so in the oral cavity.

The orally disintegrating or dissolving delivery systems are known in the art. One such commercially marketed delivery system is based on proprietary Zydis® technology (Scherer). This system is based on tablet-shaped freeze-dried solid gelatine or starch matrix network also comprising a water-soluble sugar, such as mannitol. Despite the tablet appearance, such form is actually not made by tabletting, but is a wafer made by freeze drying of a solution of ingredients in a tablet-shaped “pocket.” Such technology is complicated and expensive, requiring special equipment. Similar technologies based on freeze-drying are Lyoc technology (L. Lafon) or QuickSolv technology (Janssen).

Orally disintegrating tablets produced by tabletting are also known. In general, the fast disintegrating/dissolving attribute is achieved by facilitating quick ingress of water into the tablet matrix. The basic approaches for making such tablet include maximizing the porous structure of the tablet matrix, incorporating appropriate disintegrating agents, and using highly water-soluble excipients such as sugars or alcohols. Many of the commercial orally dissolving tablets use specifically pre-treated excipients.

One system is Flash Dose technology (Fuisz) in which tablets are made by compressing microparticles of a drug and a cotton candy-like fibrous saccharide matrix (a “floss”). This system requires specific equipment for making the matrix, is sensitive to moisture, and generally results in tablets of high friability.

OraSolv technology (Cima) involves effervescent, microencapsulated tablets. This technique requires specific package technology due to softness and friability of the tablet.

An example of a fast-dissolving conventional tablet is based on Wowtab technology (Yamanouchi), which is a conventionally processed and packed tablet based on a combination of low and high moldability saccharides as tablet excipients (U.S. Pat. No. 5,576,014).

Another example is FlashTab technology (Prographarm), which comprises coated microparticles of the active substance (to suppress the unpleasant taste) with excipients. See U.S. Pat. Nos. 5,464,632 and 6,106,861. In U.S. Pat. No. 6,106,861, for example, a disintegrant and a specific class of water soluble diluent are used to effect oral disintegration properties.

The above techniques tend to require special manufacturing and/or produce tablets that are problematic in terms of water sensitivity, hardness or friability. It would be desirable to have an oral disintegrating tablet that can be made with low friability and by ordinary tabletting techniques.

Separate from oral disintegration concerns, microcrystalline cellulose has been used as a binder especially in direct compression tablet formulations. A modified form of microcrystalline cellulose is taught in U.S. Pat. No. 5,585,115 wherein the microcrystalline cellulose is coprocessed with silicon dioxide to form an intimate mixture. Such a modified cellulose is referred to as silicified microcrystalline cellulose. According to U.S. Pat. No. 5,585,115 silicified microcrystalline cellulose has enhanced compressibility properties, especially in wet granulation conditions, thereby making it more attractive as a binder or diluent in a greater variety of tablet forming processes. Silicified microcrystalline cellulose is commercially available from Penwest under the trade name PROSOLV.

Silicified microcrystalline cellulose has been used to improve certain formulations. For example, WO 99/15155 teaches a pharmaceutical preparation comprising clodronate as the active and silicified microcrystalline cellulose as the excipient. Such compositions can provide good tablet strength, friability, compressibility, and higher loading of the clodronate. No mention is made in WO 99/15155 of disintegration times or achieving oral disintegration.

Similarly, U.S. Pat. No. 6,190,696 teaches a thyroxine formulation containing a stabilizer. Microcrystalline cellulose, especially silicified microcrystalline cellulose, is taught to enhance the stability of the formulation. More recently published U.S. patent application 20030050312 teaches forming tablets and capsules having low amounts of active, such as less than 3%, by using a mixture of microcrystalline cellulose and silicon dioxide, preferably silicified microcrystalline cellulose. The excipient is reported to increase the homogeneity of the blend. Again, neither of these patent disclosures mentions oral disintegration.

It would be desirable to provide an alternative orally disintegrating tablet having adequate disintegratability and solubility in the oral cavity and sufficient mechanical strength, e.g., to resist destruction in the course of manufacture, storage, transport, and/or use.

### SUMMARY OF THE INVENTION

The present invention is based on the surprising discovery that orally disintegrating tablets may be made from water insoluble tablet matrix-forming excipients. Accordingly, a first aspect of the invention relates to an orally disintegratable pharmaceutical tablet which comprises an effective amount of a pharmaceutically active agent and at least 50% silicified microcrystalline cellulose. The tablet disintegrates in less than 90 seconds, preferably 60 seconds or less, more preferably 30 seconds or less. The tablet optionally contains a disintegrant such as low substituted hydroxypropyl cellulose. The tablets can have conventional hardness, such as 20N to 50N, and low friability, such as 1% or less, while being easily manufactured by conventional techniques. A preferred embodiment relates to an orally disintegrating tablet which disintegrates in 30 seconds or less and which comprises an active agent, the improvement of which comprises providing a matrix of silicified microcrystalline cellulose in an amount of at least 50% within the tablet. Another preferred embodiment relates to a pharmaceutical orally disintegratable tablet which consists essentially of 50% to 90% silicified microcrystalline cellulose, 0% to 20% of low substituted hydroxypropyl cellulose, a lubricant, and an effective amount of a pharmaceutically active agent, wherein the tablet exhibits disintegration within 1 to 15 seconds when tested in an in vitro disintegration test.

Another aspect of the invention relates to the use of silicified microcrystalline cellulose in making an orally disintegrating tablet.

A further aspect of the invention relates to a process of rapidly releasing an active agent from a solid tablet, which comprises disintegrating a tablet, which comprises at least 50% of a matrix of silicified microcrystalline cellulose and an effective amount of an active agent, by placing the tablet in a water environment for up to 30 seconds.

### DESCRIPTION OF THE INVENTION

The present invention relates to the surprising discovery that silicified microcrystalline cellulose can be used to provide an orally disintegrable tablet. This ability was not known from the above-recited prior patent disclosures. Indeed, because silicified microcrystalline cellulose is a water insoluble tablet matrix-forming excipient, the use thereof in providing oral disintegration is contrary to the conventional approach in the art for oral disintegration tablets. The orally disintegrable tablets of the present invention include silicified microcrystalline cellulose as a matrix-forming excipient, typically in an amount of at least 50%, typically 50% to 90%, more typically 60% to 80%, of the total tablet mass. In some embodiments, high relative amounts of silicified microcrystalline cellulose are used such as 85-99%, more typically 90-98%, based on the total tablet mass.

Various embodiments of the orally disintegrating tablets of the present invention may provide one or more of the following features:

compressible by a known tablet press and packable in a known package;

portable without fragility concerns, having low friability;

low sensitivity to environmental conditions such as moisture and temperature;

able to load a high amount of the drug, resulting in smaller tablet size; and

leave no or minimal residue in the mouth, have pleasant mouth feel, and be compatible with taste masking.

“Orally disintegratable” means that the tablet disintegrates or disperses within 90 seconds as measured by the in vitro disintegration test described in US Pharmacopoeia 701, without disks. Such a disintegration test result is reasonably related to the actual disintegration time experienced by a mammal when placed in the oral cavity (albeit placement within such a cavity is not required). Generally, though not necessarily, the in vitro time is somewhat longer than the orally experienced time for disintegration. The disintegration of the tablet means that the tablet shape/form is destroyed but does not necessarily mean that the entire tablet is dissolved. For example, insoluble fragments can remain. In general no residue remains on the screen, which has 2 mm mesh size, or only a soft mass having no palpably firm core remains. If coated particles of the active agent are contained within the tablet, as described hereinafter, such particles can be present on the screen and need not further disintegrate, although typically such particles are too small to be held by the screen mesh and thus are also not present as a residue on the screen. Preferably, the tablets of the present invention disintegrate in less than 80 seconds, more preferably less than 60 seconds including less than 50 seconds and even less than 40 seconds, and most preferably in less than 30 seconds. In some embodiments, the disintegration is not instantaneous, but rather takes at least 0.5 seconds, more preferably at least 2 seconds. In some preferred embodiments, the disintegration occurs within the range of 1 to 30 seconds, more preferably 1 to 20 seconds, still more preferably 1 to 15 seconds, and frequently within 1 to 10 seconds. It should be noted that the corresponding European Pharmacopoeia method generally provides similar results to the above-quoted USP method.

The silicified microcrystalline cellulose (referred sometimes herein under as “silicified cellulose”) is an intimate physical mixture of colloidal silicon dioxide with microcrystalline cellulose as described in U.S. Pat. No. 5,585,115. It is not merely an admixture, but rather an intimate mixture usually formed by mixing the silicon dioxide with a suspension or slurry of microcrystalline cellulose and drying the mixture, such as by spray drying. The amount of silicon dioxide is normally within the range of 0.1 to 20 wt %, preferably from about 0.5 to 10 wt %, more typically from 1.25 to 5 wt %, and conveniently about 2 wt %, based on the weight of the silicified cellulose. The silicon dioxide generally has an average particle size not greater than 100 microns and typically between 5 and 50 microns. The microcrystalline cellulose is not particularly limited and generally has an average particle size in the range of 20 to 200 microns. For example, ProSolv 50 and ProSolv 90 (Penwest) are commercially available silicified (2% Si) microcrystalline celluloses having a median particle size of 50 and 90 microns, respectively, and are conveniently used in the present invention. Surprisingly, ProSolv 50 generally has an inferior taste/feeling in the mouth in comparison to ProSolv 90. Thus, silicified microcrystalline cellulose having a median particle size in the range of 75 to 125, especially about 90 microns, are likely preferred from this perspective.

In the tablet of the invention, the disintegration property of silicified cellulose may be enhanced by the presence of a traditional gastric disintegrant. Although such an auxiliary excipient is not necessary, the presence of a disintegrant allows for more homogeneous splitting and breaking of the tablets, a broader range of tablet compaction conditions, and higher loading of the active substance that otherwise may negatively affect the disintegration rate. Generally the amount of disintegrant is within the range of 0 to 20%. When the disintegrant is present, it is typically contained in an amount of 0.1% to 20%, more typically from 0.5% to 15%, still more typically 0.5% to 10% of the tablet mass.

An example of the disintegrant is an hydroxypropyl cellulose (HPC), especially low substituted hydroxypropyl cellulose (L-HPC) as defined in USP. Other suitable disintegrants include sodium starch glycollate, carboxymethyl cellulose, crosscarmellose sodium, crosspovidone, and starch. The disintegrant may be water soluble or insoluble, but is typically water swellable, which accounts for its disintegrating ability. The disintegrant may be non-hygroscopic. Preferably the disintegrant is not water soluble.

Another excipient that can affect the oral disintegration is a lubricant. A preferred lubricant that tends to facilitate faster disintegration rates is sodium stearyl fumarate, although other lubricants such as magnesium stearate can be used as well. In general, the lubricant should be hydrophilic.

Another factor affecting the disintegration is the tablet hardness and/or the compaction force used in making the tablet hardness. The hardness of the tablet has an influence on the disintegration time as it affects the porosity of the matrix and, accordingly, the ability of water to penetrate through the matrix. Generally, the hardness may range from 10 to 50 N, but typically is about 30 N or less. A higher hardness is preferred from the standpoint of handling and thus a hardness of at least 20 N is normally preferred. However, lower hardness generally increases disintegration speed and thus a hardness not greater than 40 N, and more preferably not greater than 30N is normally preferred. Thus, a preferred range is generally from 20 to 40 N. If porosity is sufficiently high, water can easily penetrate the tablet. Accordingly, a suitable hardness depends in part on the tablet composition and the desired level of oral disintegration speed.

The size and shape of the tablet can also affect the disintegration time. In general a smaller tablet, in terms of mass, has a faster disintegration time than a larger tablet, all other factors being equal. Similarly, a tablet shape with more surface area generally has a faster disintegration time than a tablet shape having less surface area, all other factors being equal. For pharmaceutical tablets, the weight is generally about 150 mg or less, typically about 100 mg or less, and in some embodiments about 80 mg or less, including 50 mg, although much higher tablet weights can be used such as up to 560 mg. In pharmaceutical tablets it is preferred that the pharmaceutically active agent and the silicified cellulose account for at least 80%, preferably at least 85%, more preferably at least 90% of the tablet mass. The shape of a tablet includes round, oval, and polygonal, e.g. pentagonal, octagonal, etc., which can be flat or biconvex. Additionally, the tablet may be scored and/or inscribed. Round tablets and oval tablets generally have a diameter or length, respectively, of 20 mm or less, such as 5 to 20 mm, more typically 5 to 10 mm, such as 8 mm, 6 mm, or 5 mm, but are not limited thereto.

Due to the presence of silicified cellulose, the friability of the tablet is generally less than 1.0%, such as less than 0.5%, or less than 0.2%, as measured according to European Pharmacopoeia 2.9.7.

Additional auxiliary excipients, which may have no or almost no influence on the disintegration properties, may be present in the tablet composition. Examples of auxiliary excipients include taste masking agents, stabilizers, natural or artificial sweeteners (e.g., aspartame), flavors (e.g., mint flavor), preservatives, and pH adjustors. Other auxiliary excipients may be used in case of need. Water-soluble fillers and binders, commonly used in other orally disintegrating tablets, such as sugars, sugar alcohols, or polyols (e.g., mannitol), are not required to be present and are preferably excluded. They may be present in small amounts, e.g. generally less than 5%, preferably less than 1%, and most preferably 0%. Indeed, in a preferred embodiment, water soluble excipients of any kind, are limited to be not more than 10%, more preferably not more than 5%, more typically not more than 3%, and in some embodiments are 0%, of the total mass of the tablet.

Similarly, effervescent excipients like calcium carbonates, are not required to be present in the inventive composition and are preferably excluded therefrom. The term effervescent excipient includes compounds that evolve gas. For instance, effervescent couples evolve gas by means of chemical reactions that take place upon exposure of the effervescent couple to water and/or to saliva in the mouth. The bubble or gas generating reaction is most often the result of the reaction of a soluble acid source and alkali metal carbonate or carbonate source. The reaction of these two general classes of compounds produces carbon dioxide gas upon contact with water included in saliva.

The silicified cellulose can exhibit a gritty feeling, albeit not unpleasant, in the mouth after disintegration. By itself, it has no taste and conventional sweeteners or flavors may be used to mask unpleasant tastes that could be caused by the active agent. If such a taste is not masked, the active substance may be pre-treated before adding it into the tablet matrix by measures known in the art, such as by micro- or nano-encapsulation within a coat.

There is no limitation on the active agent useful in the rapid dispersible tablets of the invention. The active substance may be a water-soluble or water-insoluble substance. It may be used in solid, particulate, granular, crystalline, amorphous, or oily form. Generally the active agent is a pharmaceutically active agent, a nutrient, a nutraceutical, or a cosmetic. A nutrient includes food and food additives. A nutraceutical includes vitamins, enzymes, proteins, etc. that provide a beneficial effect. Whenever appropriate, particles of the active agent, optionally granulated with other excipients, may be coated. For example, a suitable coating (or similarly treatment) for masking an unpleasant taste, and/or for preventing too early absorption of the drug, e.g., by oral mucosa, and/or for controlling the release or absorption of the drug in body fluids can be applied using compositions and techniques known in the art. In particular enteric coatings and extended release coatings can be used to provide an orally disintegratable tablet that provides sustained and/or controlled release of the active agent.

There is no limitation on the therapeutic class of the active ingredient. Examples of the therapeutic classes of pharmaceutical active agent include:

antipyretic/analgesic/anti-inflammatory agent,

antipsychotic/antidepressant agent,

hypnotic/sedative agent,

gastrointestinal function conditioning agent,

antitussive agent,

antihypertensive/cardiovascular system conditioning agent,

antiasthmatic/antiallergic agent,

antiparkinskonic/anti-Alzheimer agent,

hypolipidemic agents,

antimicrobial or antiviral agents, and

chemotherapy/chemotherapeutic agents.

The tablets of the invention may also comprise two or more active components, from the same or different therapeutic category and/or active agent category.

There are a number of drug candidates that are ideal for delivery via orally disintegrating dosage forms. Examples include:

fast-acting medications (e.g., drugs for treating pain, inflammation, migraine, angina, asthma, ulcers, diarrhea, or anxiety)

compliance-critical medications (e.g., drugs for cardiovascular diseases, hypertension, Parkinson's disease, psychosis, and seizures)

pediatric medications (e.g., cough/cold/allergy products, analgesics, antipyretics, and antibiotics)

Illustrative and non-limiting examples of pharmaceutical active ingredients for making tablets of the invention, alone in a combination, include: ibuprofen, acetaminophen, piroxicam (anti-inflammatory), leflunomide (antirheumatics), ondansetron, granisetron (antiemetics), paracetamol (analgetic), carbamazepin, lamotrigine (antiepileptic), clozapine, olanzapine, risperidone, citalopram, paroxetine, sertraline, fluoxetine, fluvoxamine (antipsychotics/antidepressants), zopiclon, zolpidem (hypnotics), cimetidine, ranitidine, omeprazole (antiulceric), metoclopramide, cisapride, domperidon (prokinetic), zafirlukast, montelukast (antiasthmatics), pramipexole, selegiline (anti-parkinsonics), donepezil (anti-alzheimers), zolpidem, zopiclon (hypnotics), doxazosin, terazosin, atenolol, bisoprolol, amlodipine, nifedipine, diltiazem, enalapril, captopril, ramipril, losartan (cardiovasculars), glyceroltrinitrate (vasodilantant), alfuzosin, finasteride (urologic), pravastatin, atorvastatin, simvastatin, gemfibrozil (hypolipidemics), metformin, pioglitazone (antidiabetic), terfenadine, loratadine (antihistaminic), celecoxib, rofecoxib, rivastigmine

Wherever appropriate or possible, the active agent can be used as a pharmaceutically acceptable salt or ester of the base compound and includes hydrates and solvates thereof. The invention is also not limited to a particular polymorph or enantiomer of such active ingredient.

The amount of the active ingredient in a single tablet is generally effective for its intended purpose. Usually an effective amount is within the range of 0.01 to 100 mg, more typically 0.1 to 40 mg, especially 1 to 20 mg. In relative terms, the active agent is generally present from 0.01 to 50% of the tablet mass, preferably 1 to 30%, more typically 5 to 20%.

A preferred class of tablets has the following recipe:

| | %/Tablet |
|---|---|
| Active agent | X |
| (preferably pharmaceutical active agent) | |
| Silicified microcrystalline cellulose | 90.5 − X |
| L-HPC | 5.0 |
| Sweetener | 2.0 |
| (e.g. aspartame) | |
| Flavorant | 2.0 |
| (e.g. mint flavor) | |
| Lubricant | 0.5 |
| (e.g. sodium stearyl fumarate) | |
| Tablet weight | 100.0 |

The tablets of the present invention can be made from ingredients that are known, commercially available or readily obtainable, via known or analogous synthetic routes, using techniques generally known in the art. In general, any tabletting method can be used for making the orally disintegrating tablets of the invention. As mentioned above, the tabletting technique should produce an appropriate hardness for the composition, weight, shape, etc. of the tablet so as to allow for oral disintegration. The tablets may be made by dry granulation, wet granulation, or direct compression.

Direct compression is technically simple and economically advantageous. No specific pretreatment step is necessary to modify the properties of the tablet matrix-forming disintegrable components before compression into a tablet. Direct compression may involve direct compression of a homogeneous mixture of the components. The homogenization of the mixture may be made without the aid of a solvent. Also, the ingredients need not be subjected to enhanced temperature during the homogenization. The active agent may be subjected to a suitable pre-treatment. For example, the active agent may be subjected to granulation, especially wet granulation, in order to improve the flowability and/or compression properties. Generally such a granulate further comprises an excipient such as a binder e.g. polyvinylpyrrolidone (PVP), but an excipient is not required. As a pre-treatment, the granulate of the active agent typically does not contain any silicified microcrystalline cellulose, although small amounts can be used. Another pre-treatment of the active agent, with or without granulation, is to coat the active agent to provide for taste masking, improved stability, modified release rate, and/or an enteric coat. Pre-treating for taste masking is particularly useful in tablets that contain olanzapine, paroxetine, zolpidem, montelukast, pioglitazone, donepezil, amlodipine, or anastrozole as the active agent. For taste masking, any conventional taste masking coating can be used including a coating comprising one or more polymers such as acrylic polymers (e.g. Eudragits) or waxes (e.g. Precirol or Compritol from Gattefosse), but is not limited thereto. For the other purposes, any conventional coatings for stability, controlled release, and/or enteric coatings can be used. The coating can be applied by any suitable technique/apparatus including pan coating or a fluid bed system, etc. Such a pre-treated active agent, e.g. granulated, coated or both, can be used in a direct compression method by combining it with the silicified microcrystalline cellulose and other excipients, if any, in one or more steps to form a tablet blend and compressing into tablets.

Wet granulation can also be used to make the tablets of the invention wherein the active agent is wet granulated with all or most of the silicified microcrystalline cellulose to form granules. The granules are mixed with the remaining excipients, typically a lubricant and any remaining silicified microcrystalline cellulose, to form a tablet blend and then compressed into tablets. Typically in a wet granulation process, all of the silicified microcrystalline cellulose is within the granulate and no extra-granular silicified microcrystalline cellulose is employed. This is in contrast to the direct compression methods wherein even if a wet granulation pre-treatment is used, most and preferably all of the silicified microcrystalline cellulose is extra-granular; i.e. not used in the pretreatment.

Depending upon the size and shape, the tablet may advantageously be made under a compression force of below 5 kg/cm², such as below about 4 kg/cm², or below 3 kg/cm².

The tablet making process results in a binder matrix of silicified cellulose having the active agent dispersed therein. The process of making the tablet composition does not require the use of compounds or processes for improving the porosity or permeability of the tablet matrix. Thus, pore forming agents, foaming agents, or similar tools may or may not be used in making tablet compositions of the invention.

Many types of patients can benefit from orally disintegrating dosage forms, such as pediatric patients, psychiatric patients, patients with renal disorders or patients with swallowing disorders. Dysphagia or difficulty in swallowing is seen to afflict nearly 35% of the general population.

In addition to ease of delivery, another potential advantage of orally disintegrating dosage forms is that they can improve the overall clinical performance of a drug by reducing the incidence of non-compliance.

The rapidly disintegrating tablets of the invention can provide a process for quickly releasing the active agent from a solid tablet. Specifically, in a preferred embodiment, the tablets can be used by placing them in a water environment for up to 30 seconds. In 30 seconds or less the tablet is disintegrated in the water environment, i.e. the tablet is no longer in existence or present in the water environment, albeit a residue thereof may be present. The destruction of the tablet allows the release of the active agent; i.e. as a per se compound, as a particle such as a coated particle, etc., as discussed above for forms of the active agent. The water environment can be any moist environment including an oral cavity, a container of water such as the disintegration apparatus or a glass of water, etc. In case of a glass of water or other similar water container, a patient may consume the product after, or even during, disintegration. In this way, the once solid dosage form is consumed as essentially a liquid, including a suspension or slurry. It is surprising that a solid tablet containing silicified cellulose could be disintegrated by contacting it with water for 30 seconds or less as the use of silicified cellulose as a rapid disintegrant and/or oral disintegrant is not described in the above-mentioned patent disclosures.

When administering the tablet to an animal, one or more tablets may be used in order to achieve the intended dose of the active agent. Such multiple tablets can be given simultaneously or sequentially, normally within a few minutes of each other.

The disclosure in each of the above-mentioned patents and published patent applications is incorporated herein in its entirety. The present invention will be further illustrated by way of the following Examples. These Examples are non-limiting and do not restrict the scope of the invention.

### EXAMPLES

Unless otherwise specified, ProSolv 90 (Penwest) was used as the silicified microcrystalline cellulose in each of the following examples.

### Example 1

Orally Disintegrating Tablets Containing Leflunomide

The composition of this Example is shown in Table 1, below.

TABLE 1

| Example 1 | mg/tablet | %/tablet |
|---|---|---|
| Leflunomide | 20.0 | 20.00 |
| Silicified microcrystalline cellulose | 74.5 | 74.50 |
| L-HPC (low substituted | 5.0 | 5.00 |
| hydroxypropylcellulose) | | |
| Magnesium stearate | 0.5 | 0.50 |
| Tablet weight | 100.0 | 100.00 |

Leflunomide, silicified microcrystalline cellulose, and L-HPC were homogeneously mixed with a Turbula mixer. The magnesium stearate was added and mixing was finalized. 6 mm round biconvex tablets were compressed in a tablet press to a hardness of 46 N.

The friability of the tablets was well below 1.0%.

The disintegration time as measured with the USP disintegration apparatus was less than 10 seconds.

### Example 2-3

Both examples were prepared as described in Example 1, except that the composition was modified as discussed below and the tablet punch was changed to an oval, biconvex tablet punch with a length of 6 mm and having an inscription “ABO” therein.

### Example 2

Leflunomide Orally Disintegrating Tablet with Sodium Stearyl Fumarate

The composition of this Example is shown in Table 2, below.

TABLE 2

| | mg/tablet | %/tablet |
|---|---|---|
| Leflunomide | 10.00 | 20.00 |
| Silicified microcrystalline cellulose | 37.75 | 74.50 |
| L-HPC | 2.50 | 5.00 |
| Sodium stearyl fumarate | 0.25 | 0.50 |
| Tablet weight | 50.0 | 100.00 |

In this case the disintegration time of the tablets was very quick. In 5 seconds the tablets had disappeared in the disintegration test.

### Example 3

Leflunomide Orally Disintegrating Tablet with Double L-HPC

The composition of this Example is shown in Table 3, below.

TABLE 3

| | mg/tablet | %/tablet |
|---|---|---|
| Leflunomide | 20.0 | 20.00 |
| Silicified microcrystalline cellulose | 69.5 | 69.50 |
| L-HPC | 10.0 | 10.00 |
| Sodium stearyl fumarate | 0.5 | 0.50 |

The disintegration time of the tablets was extremely quick. In 1-2 seconds the tablets had disappeared.

### Example 4

Orally Disintegrating Tablet Containing Ondansetron

The composition of this Example is shown in Table 4, below.

TABLE 4

| | mg/tablet | %/tablet |
|---|---|---|
| Ondansetron base | 8.00 | 13.9 |
| Silicified microcrystalline cellulose | 37.50 | 65.4 |
| L-HPC | 3.50 | 6.1 |
| Aspartame | 7.70 | 13.4 |
| mint flavor | 0.40 | 0.6 |
| Sodium stearyl fumarate | 0.25 | 0.4 |
| Tablet weight | 57.35 | 100.0 |

The ondansetron base, silicified microcrystalline cellulose, L-HPC, aspartame, and mint flavor were mixed for 15 minutes in a Turbula mixer. The sodium stearyl fumarate was added, and the mixture was mixed for 5 minutes. The tablets were pressed using a Korsch EK0 tablet press at various compression forces. The disintegration time was directly dependent on the hardness. The tablets having a hardness within the range of 10-40 N fulfilled the desirable fast disintegration criteria. The friability of the 10-40 hardness tablets was still close to 0%. The taste of the active ingredient and the gritty feel of the silicified microcrystalline cellulose was counteracted by the aspartame and mint.

### Example 5

Orally Disintegrating Tablet Containing Ondansetron Free Base

The composition of this Example is shown in Table 5, below.

TABLE 5

| | mg/tablet | %/tablet |
|---|---|---|
| Ondansetron base | 8.00 | 8.00 |
| Silicified microcrystalline cellulose | 82.50 | 82.50 |
| L-HPC | 5.00 | 5.00 |
| Aspartame | 2.00 | 2.00 |
| mint flavour | 2.00 | 2.00 |
| Sodium stearyl fumarate | 0.50 | 0.50 |
| Tablet weight | 100.00 | 100.00 |

The ondansetron base, silicified microcrystalline cellulose, L-HPC, aspartame and mint flavor were mixed for 15 minutes in a Turbula mixer. The sodium stearyl fumarate was added, and the mixture was mixed for 5 minutes. 8 mm round biconvex tablets were compressed on a Korsch PH 106 tablet press at a target hardness of 30 N. During compression no problems were observed. The tablets dispersed within 30 seconds when placed in the mouth.

### Example 6-14

Orally Disintegrating Tablets Containing a Range of Actives

In the following examples the same concept was applied to various actives, differing in solubility, dose, and/or therapeutic area.

The generic formula for all cases is shown in Table 6, below:

TABLE 6

| | %/tablet |
|---|---|
| Active drug substance | X |
| Silicified microcrystalline cellulose | 90.5 − X |
| L-HPC | 5.00 |
| Aspartame | 2.00 |
| mint flavor | 2.00 |
| Sodium stearyl fumarate | 0.50 |
| Tablet weight | 100.00 |

X = amount of drug substance used.

The manufacturing procedure for all was similar. The active drug substance, silicified microcrystalline cellulose, L-HPC, aspartame and mint flavor were mixed for 15 minutes in a Turbula mixer. The sodium stearyl fumarate was added, and the mixture was mixed for 5 minutes. In all cases, 8 mm round biconvex tablets were pressed using a Korsch EK0. Tablet hardness is 30 N, friability below 1.0%.

### Example 6

Orally Disintegrating Tablet Containing Olanzapine

Orally disintegrating tablets containing 20 mg of olanzapine and 70.5 mg of silicified microcrystalline cellulose were prepared following the above general instructions. The disintegration time in the mouth of the product was less than 30 seconds.

### Example 7

Orally Disintegrating Tablet Containing Montelukast Sodium

Orally disintegrating tablets containing 10.4 mg of montelukast sodium were prepared following the instructions as described above. A disintegration test with the Ph. Eur apparatus showed that the tablets disintegrated within 30 seconds.

### Example 8

Orally Disintegrating Tablets Containing Risperidone Free Base

Orally disintegrating tablets were prepared by following the general instructions described above. 4 mg of risperidone base was incorporated in the formula. The disintegration of the tablet in the mouth took less than 30 seconds. Also, the bitter taste of risperidone was masked by the mint and aspartame present in the formula.

### Example 9

Orally Disintegrating Tablets Containing Pramipexole

Orally disintegrating tablets containing 1.5 mg of pramipexole dihydrochloride were prepared by following the general instructions. The tablets disintegrated within 30 seconds when placed in the mouth.

### Example 10

Orally Disintegrating Tablet Containing Alendronate Sodium.

Orally disintegrating tablets containing 13.05 mg of alendronate sodium trihydrate were prepared by following the general instructions presented above. The disintegration time of the tablets as measured by the Ph. Eur. method was less than 1 minute.

### Examples 11 and 12

Orally Disintegrating Tablets Containing 10 mg Amlodipine (Calculated as Base)

Orally disintegrating tablets were made containing 10 mg amlodipine base following the general instructions as presented above with two different amlodipine salts, i.e., 14.28 mg amlodipine besylate monohydrate (Example 11) and 12.8 mg amlodipine maleate (Example 12). In both cases the disintegration time in the mouth was less than 30 seconds.

### Example 13 and 14

Orally Disintegrating Tablets Containing 2.5 mg Amlodipine (Calculated as Base)

From the blends as described in Examples 11 and 12, orally disintegrating tablets were prepared containing 2.5 mg of amlodipine (calculated as base). These tablets weighed 25 mg and they disintegrated within 30 seconds after administration.

### Example 15

Orally Disintegrating Tablets Containing Pre-coated Paroxetine Mesylate for Controlled Release Purposes

The composition of this Example is shown in Table 7, below.

TABLE 7

| | mg/tablet | %/tablet |
|---|---|---|
| Paroxetine mesylate | 25.83 | 17.22 |
| Eudragit NE 30 D | 10.00 | 6.66 |
| Silicified microcrystalline cellulose | 104.67 | 69.78 |
| L-HPC | 5.00 | 3.33 |
| aspartame | 2.00 | 1.33 |
| mint flavor | 2.00 | 1.33 |
| Sodium stearyl fumarate | 0.50 | 0.33 |
| Tablet weight | 150.00 | 100.00 |

Paroxetine mesylate was coated with Eudragit NE 30 D in a fluid bed dryer. The coated particles were mixed with the silicified microcrystalline cellulose, L-HPC, aspartame, and mint flavor in a free-fall mixer. After addition of the sodium stearyl fumarate the mixing was finalized. Oval biconvex tablets with a length of 8 mm were prepared on an EK0 tablet press. The disintegration time of the tablets as measured by the Ph. Eur. disintegration test was less than 30 seconds. The coated particles remained intact.

### Example 16

Orally Disintegrating Tablets Containing Simvastatin

The composition of this Example is shown in Table 8, below.

TABLE 8

| | mg/tablet |
|---|---|
| Simvastatin | 10.00 |
| BHA | 0.02 |
| Sodium starch glycolate | 0.34 |
| Povidon | 0.66 |
| Silicified microcrystalline cellulose | 49.46 |
| L-HPC | 4.20 |
| aspartame | 2.10 |
| mint flavor | 2.10 |
| Sodium stearyl fumarate | 1.05 |
| Iron oxide yellow | 0.07 |
| Tablet weight | 70 |

Simvastatin was granulated with BHA, sodium starch glycollate, and Povidon (PVP) as binder. The granulates are subsequently sieved and dried. The dried granulate was mixed with the silicified microcrystalline cellulose, L-HPC, aspartame, mint flavor and iron oxide yellow in a free-fall mixer. After addition of the sodium stearyl fumarate the mixing was finalized. Oval biconvex tablets with a diameter of 7 mm were prepared on an EK0 tablet press. The disintegration time of the tablets as measured by the Ph. Eur. disintegration test was less than 30 seconds.

### Example 17

Orally Disintegrating Tablets Comprising Risperidone

| | mg/tablet |
|---|---|
| Risperidone free base | 3.0 |
| Prosolv HD-90 | 78.90 |
| L-HPC | 5.0 |
| Aspartame | 6.0 |
| Mint flavour | 6.0 |
| Acesulfam K | 0.5 |
| Iron oxide red | 0.10 |
| Sodium Stearyl Fumarate | 0.5 |

After sieving the iron oxide through a 100 μm sieve, mix risperidone free base, 30% of the Prosolv, L-HPC, Aspartame, Mint flavour, Acesulfame-K and sieved iron oxide in a turbula mixer (22 rpm, 15 min). Then add 70% of the Prosolv and mix for another 15 min at 22 rpm. Sieve sodium stearyl fumarate through an 800 μm sieve and add the sieved sodium stearyl fumarate and mix for another 5 min at 22 rpm. Compress 100 mg 8 mm tablets at 30-40 N on the Korsch EK-0. Tablets disintegrate within 30 seconds.

### Example 18

Orally Disintegrating Tablets Comprising Risperidone

Tablets can be made according to the following formulation:

| | mg/tablet |
|---|---|
| Risperidone base | 4.0 |
| Silicified Microcrystalline Cellulose | 78.0 |
| L-HPC | 5.0 |
| Aspartame | 6.0 |
| Mint Flavor | 6.0 |
| Acesulfam K | 0.5 |
| Sodium Stearyl Fumarate | 0.5 |

Tablets are made by mixing the risperidone, aspartame, mint flavor, Acesulfam K, and half of the silicified microcrystalline cellulose in a free fall mixer. Add the second half of the silicified microcrystalline cellulose and mix again. Add the sodium stearyl fumarate and mix again. Compress 8 mm tablets of an average weight of 100 mg and an average hardness between 30 and 40 N.

### Example 19

Orally Disintegrating Tablets Containing Paroxetine Mesylate

| Granulate | % |
|---|---|
| Paroxetine mesylate | 10 |
| Prosolv 90HD | 71 |
| PVP | 6 |
| Explotab | 3 |
| carrageenan 911 | 10 |

All ingredients were mixed, granulated and dried in the high shear granulator.

| Pretabletting blend | mg/tablet |
|---|---|
| granulate | 90.9 |
| L-HPC | 4.55 |
| Mint flavouring spray dried (powder) | 1.8 |
| Aspartame Powder | 2.3 |
| Sodium Stearyl Fumarate | 0.45 |

Mix the sieved granules with L-HPC, mint and aspartame in a Turbula mixer for 20 minutes at 22 rpm. Add the sodium stearyl fumarate and mix for 5 minutes at 22 rpm. Compress tablets using 8 mm punch on EK-0. Target tablet weight=100 mg. Tablet hardness 30N. Tablets disintegrate within 30 seconds.

### Example 20

Orally Disintegrating Tablets Containing Donepezil

| Granulate | % |
|---|---|
| Donepezil-hydrochloride | 5 |
| Prosolv HD-90 | 78 |
| PVP | 6 |
| Explotab | 6 |
| Carrageenan 812 | 5 |

All ingredients were mixed, granulated and dried in the high shear granulator.

| Pretabletting blend | mg/tablet |
|---|---|
| granulate | 90.9 |
| L-HPC | 4.55 |
| Mint flavouring spray dried (powder) | 1.8 |
| Aspartame Powder | 2.3 |
| Sodium Stearyl Fumarate | 0.45 |

Mix the sieved granules with L-HPC, mint and aspartame in a Turbula mixer for 20 minutes at 22 rpm. Add the sodium stearyl fumarate and mix for 5 minutes at 22 rpm. Compress tablets using 8 mm punch on EK-0. Target tablet weight=100 mg. Tablet hardness 30N. Tablets disintegrate within 30 seconds.

### Example 21

Orally Disintegrating Tablets Containing Zolpidem (Taste Masking):

| Granulate | mg/tablet |
|---|---|
| Zolpidem hemitartrate | 5.0 |
| Compritol | 0.5 |
| Prosolv HD-90 | 44.3 |
| L-HPC | 0.25 |
| Aspartame | 0.1 |
| Mint flavour | 0.1 |
| Sodium Stearyl Fumarate (Pruv) | 0.05 |
| Total weight | 50 |

The Zolpidem particles are coated by applying compritol via a Fluid bed process. Afterwards, the coated Zolpidem particles, Prosolv, L-HPC, aspartame and mint flavour are mixed in a free fall mixer, followed by blending the sodium stearyl fumarate. Tablets were prepared on a Korsch EK-0 tablet press at a hardness of 30 N. Tablets disintegrate within 30 seconds.

### Example 22

Orally Disintegrating Tablets Containing Tamsulosin Hydrochloride with Enteric Coating

| | mg/tablet |
|---|---|
| Tamsulosin hydrochloride | 0.25 |
| Eudragit | 0.038 |
| Triethylcitrate | 0.004 |
| Prosolv HD-90 | 49.225 |
| L-HPC | 0.25 |
| Aspartame | 0.1 |
| Mint flavour | 0.1 |
| Sodium Stearyl Fumarate (Pruv) | 0.05 |
| Total weight | 50 |

The tamsulosin particles are coated with Eudragit in a fluid bed system. The coated granules are mixed with L-HPC, mint and aspartame in a Turbula mixer for 20 minutes at 22 rpm. Add the sodium stearyl fumarate and mix for 5 minutes at 22 rpm. Compress tablets using 8 mm punch on EK-0. Target tablet weight=50 mg. Tablet hardness 30N. Tablets disintegrate within 30 seconds.

In view of the above description of the invention, it will be readily apparent to the worker skilled in the art that the same may be varied in many ways without departing from the spirit of the invention and such modifications are included within the scope of the present invention as set forth in the following claims.

We claim:

1. A tablet for oral administration, comprising an effective amount of an active agent and at least 50% silicified microcrystalline cellulose, wherein said tablet is orally disintegratable.

2. The tablet according to claim 1, wherein said tablet exhibits oral disintegratability in not more than 60 seconds.

3. The tablet according to claim 2, wherein said tablet exhibits oral disintegratability in not more than 30 seconds.

4. The tablet according to claim 1, wherein said tablet exhibits oral disintegratability in not less than 0.5 second.

5. The tablet according to claim 4, wherein said tablet exhibits oral disintegratability in not less than 2 seconds.

6. The tablet according to claim 4, wherein said tablet exhibits oral disintegratability within the range of 1 to 15 seconds.

7. The tablet according to claim 1, wherein said silicified microcrystalline cellulose is contained in an amount within the range of 55% to 90%.

8. The tablet according to claim 7, wherein said silicified microcrystalline cellulose is contained in an amount within the range of 60% to 80%.

9. The tablet according to claim 1, wherein said active agent and said silicified microcrystalline cellulose together represent at least 80% of the tablet weight.

10. The tablet according to claim 6, wherein said active agent and said silicified microcrystalline cellulose together represent at least 80% of the tablet weight.

11. The tablet according to claim 1, wherein said silicified microcrystalline cellulose contains 1-5% silicon dioxide.

12. The tablet according to claim 1, wherein said silicified microcrystalline cellulose has an average particle size within the range of 20-200 microns.

13. The tablet according to claim 12, wherein the median particle size is about 90 microns.

14. The tablet according to claim 1, which further comprises a disintegrant.

15. The tablet according to claim 14, wherein said disintegrant is selected from the group consisting of low substituted hydroxypropyl cellulose, carboxymethyl cellulose, crosscarmellose sodium, crosspovidone, starch, and combinations thereof.

16. The tablet according to claim 15, wherein said disintegrant is low substituted hydroxypropyl cellulose.

17. The tablet according to claim 14, wherein said disintegrant is contained in an amount of 0.5% to 20%.

18. The tablet according to claim 1, which does not contain an effervescent excipient.

19. The tablet according to claim 1, which has a hardness of 20N to 40N.

20. The tablet according to claim 1, which has a friability of less than 1%.

21. The tablet according to claim 1, wherein said tablet does not contain a water soluble binder.

22. The tablet according to claim 1, which further comprises at least one additional excipient selected from the group consisting of taste masking agents, sweeteners, lubricants, stabilizers, preservatives, and pH-adjustors.

23. The tablet according to claim 1, wherein said active agent is selected from the group consisting of pharmaceutical active agents, nutrients, nutraceuticals, and cosmetics.

24. The tablet according to claim 23, wherein said active agent is one or more vitamins.

25. The tablet according to claim 23, wherein said active agent is a pharmaceutically active agent.

26. The tablet according to claim 25, wherein said pharmaceutically active agent is present in the form of coated particles containing said pharmaceutically active agent.

27. The tablet according to claim 26, wherein said coating is an extended release or an enteric coating.

28. The tablet according to claim 25, wherein said pharmaceutically active agent is selected from the group consisting of anti-inflammatories, antirheumatics, antiemetics, analgetics, antiepileptics, antipsychotics, antidepressants, hypnotics, antiulcerics, prokinetic, antiasthmatics, anti-parkinsonics, cardiovasculars, vasodilators, urologics, hypolipidemics, antidiabetics, and antihistaminics.

29. The tablet according to claim 25, wherein said pharmaceutically active agent is selected from the group consisting of ibuprofen, acetaminophen, piroxicam, leflunomide, ondansetron, granisetron, paracetamol, carbamazepin, lamotrigine, clozapine, olanzapine, risperidone, citalopram, paroxetine, sertraline, fluoxetine, fluvoxamine, zopiclon, zolpidem, cimetidine, ranitidine, omeprazole, metoclopramide, cisapride, domperidon, zafirlukast, montelukast, pramipexole, donepezil, selegiline, zolpidem, zopiclon, doxazosin, terazosin, atenolol, bisoprolol, amlodipine, nifedipine, diltiazem, enalapril, captopril, ramipril, losartan, glyceroltrinitrate, alfuzosin, finasteride, pravastatin, atorvastatin, simvastatin, gemfibrozil, pioglitazone, metformin, terfenadine, loratadine, celecoxib, rofecoxib, and rivastigmine.

30. A pharmaceutical orally disintegratable tablet which consists essentially of 50% to 90% silicified microcrystalline cellulose, 0% to 20% of low substituted hydroxypropyl cellulose, a lubricant, and an effective amount of a pharmaceutically active agent, wherein said tablet exhibits disintegration within 1 to 15 seconds when tested in an in vitro disintegration test.

31. The pharmaceutical tablet according to claim 30, wherein said tablet further comprises flavorants, colorants, or both.

32. A process of rapidly releasing an active agent from a solid tablet, which comprises disintegrating a tablet, which comprises at least 50% of a matrix of silicified microcrystalline cellulose and an effective amount of an active agent, by placing the tablet in a water environment for up to 30 seconds.

33. The process according to claim 30, wherein said water environment is a water-filled container.

34. In an orally disintegrating tablet which disintegrates in 30 seconds or less and which comprises an active agent, the improvement of which comprises providing a matrix of silicified microcrystalline cellulose in an amount of at least 50% within the tablet.

35. The orally disintegrating tablet according to claim 34, which does not contain a water soluble binder.
